(19) United States
(12) Patent Application Publication
(10) Pub. No.: US 2024/0033521 A1
(43) Pub. Date: Feb. 1, 2024

(54) ELECTRICAL NERVE STIMULATION

(71) Applicants: NATIONAL CHENG KUNG UNIVERSITY, Tainan (TW); The Regents of the University of California, Oakland, CA (US)

(72) Inventors: Yuan-Yu Hsueh, TAINAN (TW); Wentai Liu, Los Angeles, CA (US); Szu Han Chen, TAINAN (TW); Wan-Ling Tseng, TAINAN (TW); CHUN-WEI LIN, TAINAN (TW)

(21) Appl. No.: 18/227,791

(22) Filed: Jul. 28, 2023

Related U.S. Application Data
(60) Provisional application No. 63/369,753, filed on Jul. 28, 2022.

(51) Int. Cl. A61N 1/36

(52) U.S. Cl. CPC A61N 1/36139; A61N 1/36171; A61N 1/36153

(57) ABSTRACT

A nerve stimulation system includes an electrode, an electrode controlling device coupled to the electrode and configured to control the electrode to electrically stimulate a peripheral nerve according to a nerve stimulation signal, and a signal generating device coupled to the electrode controlling device and configured to generate the nerve stimulation signal. The nerve stimulation signal is a signal with a square envelope. The square envelope periodically includes an on-time period with a pulse amplitude and an off-time period without the pulse amplitude, and a ratio of the on-time period and the off-time period is not less than 1, and a length of the off-time period is not longer than 5 seconds.

### CROSS-REFERENCE TO RELATED APPLICATION(S)

The present disclosure claims the benefit of and priority to U.S. Provisional Patent Application Ser. No. 63/369,753, filed on Jul. 28, 2022, entitled “ELECTRICAL NERVE STIMULATION,” the content of which is hereby incorporated fully by reference into the present disclosure.

### FIELD

The present disclosure generally relates to a stimulation system and, more particularly, to a nerve stimulation system.

### BACKGROUND

In recent years, dozens of therapeutic nerve electrical stimulation devices have been developed. The electrical stimulation devices may be used to increase activity of neuromuscular junctions (NMJ) to treat acute/chronic nerve injury such as carpal tunnel syndrome (CTS), and muscle degenerative disease such as congenital muscular dystrophy (CMD). Traditionally, the electrical stimulation treatment is performed by using a continuous nerve stimulation signal. However, it is found nonoptimal.

Acetylcholine (ACh) may be released from an axon terminal in response to an electrical stimulation performed on the NMJ, the ACh may then be absorbed by a receptor of a muscle cell. The ACh may be hydrolyzed by Acetylcholinesterase (AChE) at neuromuscular junctions into Choline and Acetate. After that, the Choline may be transported back into the axon terminal (e.g., recycle) and used to make more ACh (e.g., reproduction) in the ACh synthesis pathway. Thus, it may take some time for the recycle of the Choline and the reproduction of the Ach. Therefore, an intermittent nerve stimulation signal is beneficial for the treatment.

### SUMMARY

In the present disclosure, an optimal waveform of the nerve stimulation signal is found and a nerve stimulation system, a signal generating device, and an operation method of the nerve stimulation system adopting the optimal waveform are provided.

In a first aspect of the present disclosure, a nerve stimulation system is provided. A nerve stimulation system includes an electrode, an electrode controlling device coupled to the electrode and configured to control the electrode to electrically stimulate a peripheral nerve according to a nerve stimulation signal, and a signal generating device coupled to the electrode controlling device and configured to generate the nerve stimulation signal. The nerve stimulation signal is a signal with a square envelope. The square envelope periodically includes an on-time period with a pulse amplitude and an off-time period without the pulse amplitude. A ratio of the on-time period and the off-time period is not less than 1, and a length of the off-time period is not longer than 5 seconds.

In an implementation of the first aspect of the present disclosure, the electrode is to be implanted and aiming at a point between a lesion of the peripheral nerve and a muscle associated with the peripheral nerve.

In an implementation of the first aspect of the present disclosure, the nerve stimulation system further includes an electromyography (EMG) monitoring device coupled to the signal generating device and is configured to acquire an EMG signal associated with a muscle. In a case that a voltage of the EMG signal is greater than a maximum threshold, the signal generating device is further configured to decrease an amplitude of the nerve stimulation signal for electrically stimulating the peripheral nerve, and in a case that the voltage of the EMG signal is less than a minimum threshold, the signal generating device is further configured to increase the amplitude of the nerve stimulation signal for electrically stimulating the peripheral nerve.

In an implementation of the first aspect of the present disclosure, the maximum threshold is 800 μV.

In an implementation of the first aspect of the present disclosure, the minimum threshold is 200 μV.

In an implementation of the first aspect of the present disclosure, the nerve stimulation system further includes a prompt device coupled to the EMG monitoring device. In a case that the voltage of the EMG signal is greater than the maximum threshold, the prompt device is configured to provide a prompt indicating an excessive current. In a case that the voltage falls within a voltage range defined by the maximum threshold and the minimum threshold, the prompt device is configured to provide the prompt indicating an efficient stimulation. In a case that the voltage of the EMG signal is less than the minimum threshold, the prompt device is configured to provide the prompt indicating an insufficient current.

In an implementation of the first aspect of the present disclosure, a length of the off-time period is within a range from 0.5 second to 5 seconds.

In an implementation of the first aspect of the present disclosure, a ratio of the on-time period to the off-time period is 4.

In an implementation of the first aspect of the present disclosure, a length of the on-time period is with a range from 3 seconds to 9 seconds.

In an implementation of the first aspect of the present disclosure, a frequency of the nerve stimulation signal in the on-time period is within a range of 5 Hz to 500 Hz.

In an implementation of the first aspect of the present disclosure, a duty cycle of the on-time period and the off-time period is within a range of 60% to 80%.

In a second aspect of the present disclosure, a signal generating device is provided. A signal generating device, including a transceiver coupled to an electrode controlling device; and a processor coupled to a transceiver and configured to generate a nerve stimulation signal; and transmit, using the transceiver, the nerve stimulation signal to the electrode controlling device such that the electrode controlling device controls an electrode to electrically stimulate a peripheral nerve according to the nerve stimulation signal, where the nerve stimulation signal is a signal with a square envelope, the square envelope periodically comprises an on-time period with a pulse amplitude and an off-time period without the pulse amplitude, a ratio of the on-time period and the off-time period is not less than 1, and a length of the off-time period is not longer than 5 seconds.

In an implementation of the second aspect of the present disclosure, the electrode is implanted by aiming at a point which is located between a lesion of the peripheral nerve and a muscle associated with the peripheral nerve.

In an implementation of the second aspect of the present disclosure, the processor is further configured to receive an electromyography (EMG) signal associated with the muscle from an EMG monitoring device by using the transceiver; determine whether a voltage of the EMG signal falls within a voltage range defined by a maximum threshold and a minimum threshold; in a case of determining that the voltage of the EMG signal is greater than the maximum threshold, decrease an amplitude of the nerve stimulation signal; and in a case of determining that the voltage of the EMG signal is less than the minimum threshold, increase the amplitude of the nerve stimulation signal.

In an implementation of the second aspect of the present disclosure, a frequency of the nerve stimulation signal in the on-time period is within a range of 5 Hz to 500 Hz.

In an implementation of the second aspect of the present disclosure, the ratio of the on-time period to the off-time period is 4.

In a third aspect of the present disclosure, an operation method of a nerve stimulation system is provided. An operation method of a nerve stimulation system including generating a nerve stimulation signal by a signal generating device of the nerve stimulation system; providing an electrical stimulation according to the nerve stimulation signal via an electrode of the nerve stimulation system; acquiring an electromyography (EMG) signal associated with a subject by an EMG monitoring device of the nerve stimulation system; and determining whether a voltage of the EMG signal falls within a voltage range defined by a maximum threshold and a minimum threshold, where the nerve stimulation signal is a signal with a square envelope, the square envelope periodically comprises an on-time period with a pulse amplitude and an off-time period without the pulse amplitude, a ratio of the on-time period and the off-time period is not less than 1, and a length of the off-time period is not longer than 5 seconds.

In an implementation of the third aspect of the present disclosure, a length of the off-time period is within a range from 0.5 second to 5 seconds.

In an implementation of the third aspect of the present disclosure, the length of the off-time period is 2 seconds. In an implementation of the third aspect of the present disclosure, the ratio of the on-time period to the off-time period is 4.

### BRIEF DESCRIPTION OF THE DRAWINGS

The present disclosure will be better understood from the following detailed description read in light of the accompanying drawings, where:

FIG. 1 is a block diagram illustrating a nerve stimulation system according to an example implementation of the present disclosure.

FIG. 2 is a block diagram illustrating a signal generating device according to an example implementation of the present disclosure.

FIG. 3 is a block diagram illustrating a nerve stimulation system according to another example implementation of the present disclosure.

FIG. 4 is a schematic diagram illustrating a nerve stimulation system in operation according to an example implementation of the present disclosure.

FIG. 5 is a schematic diagram illustrating a nerve stimulation system in operation according to another example implementation of the present disclosure.

FIG. 6 is a schematic diagram illustrating a waveform of a nerve stimulation signal according to an example implementation of the present disclosure.

FIG. 7 is a schematic diagram illustrating a waveform of a nerve stimulation signal in an on-time period according to an example implementation of the present disclosure.

FIG. 8 is a flowchart illustrating an operation method of a nerve stimulation system according to an example implementation of the present disclosure.

FIG. 9 illustrates AchE relative expressions with different on:off ratios according to an example implementation of the present disclosure.

FIG. 10 illustrates aAChR mRNA relative expressions with different on:off ratio ratios according to an example implementation of the present disclosure.

FIG. 11 illustrates myo D mRNA relative expressions with different on:off ratios according to an example implementation of the present disclosure.

FIG. 12 illustrates a number of neuromuscular junctions (NMJ) with different on:off ratios according to an example implementation of the present disclosure.

FIG. 13 illustrates AchE/total proteins with different stimulation frequencies according to an example implementation of the present disclosure.

### DETAILED DESCRIPTION

The following disclosure contains specific information pertaining to exemplary implementations in the present disclosure. The drawings in the present disclosure and their accompanying detailed disclosure are directed to merely exemplary implementations. However, the present disclosure is not limited to merely these exemplary implementations. Other variations and implementations of the present disclosure will occur to those skilled in the art. Unless noted otherwise, like or corresponding elements among the figures may be indicated by like or corresponding reference numerals. Moreover, the drawings and illustrations in the present disclosure are generally not to scale and are not intended to correspond to actual relative dimensions.

For the purposes of consistency and ease of understanding, like features are identified (although, in some examples, not shown) by numerals in the exemplary figures. However, the features in different implementations may be different in other respects, and thus shall not be narrowly confined to what is shown in the figures. The disclosure uses the phrases “in one implementation,” “in some implementations,” and so on, which may each refer to one or more of the same or different implementations. The term “coupled” is defined as connected, directly or indirectly through intervening components and is not necessarily limited to physical connections. The term “comprising” means “including, but not necessarily limited to;” it specifically indicates open-ended inclusion or membership in the so-described combination, group, series and the equivalent.

Additionally, for the purposes of explanation and non-limitation, specific details, such as functional entities, techniques, protocols, standard, and the like, are set forth for providing an understanding of the described technology. In other examples, detailed disclosure of well-known methods, technologies, systems, architectures, and the like are omitted so as not to obscure the disclosure with unnecessary details.

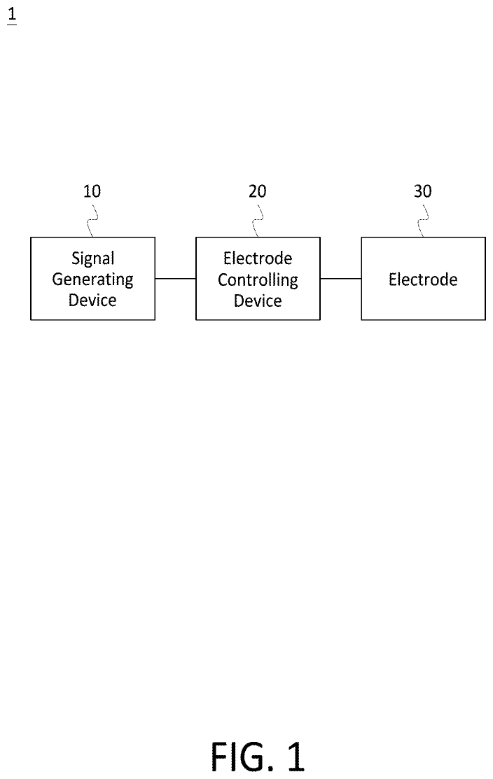

FIG. 1 is a block diagram illustrating a nerve stimulation system according to an example implementation of the present disclosure.

Referring to FIG. 1, a nerve stimulation system 1 may include a signal generating device 10, an electrode controlling device 20, and an electrode 30. The signal generating device 10 may be coupled to the electrode controlling device 20, and the electrode controlling device 20 may be coupled to the electrode 30.

In some implementations, the electrode 30 may be, for example, the “DELTA electrode” on the market manufactured by inomed® Medizintechnik GmbH. However, the present disclosure does not limit the model of the electrode 30.

In some implementations, the signal generating device 10 may be configured to generate a nerve stimulation signal in a designed waveform and transmit the generated nerve stimulation signal to the electrode controlling device 20. The electrode controlling device 20 may be configured to receive the nerve stimulation signal from the signal generating device 10 and control the electrode 30 to electrically stimulate a peripheral nerve according to the nerve stimulation signal. In some implementations, the electrode 30 may be implanted into a patient during a surgery, or using an insertion tool.

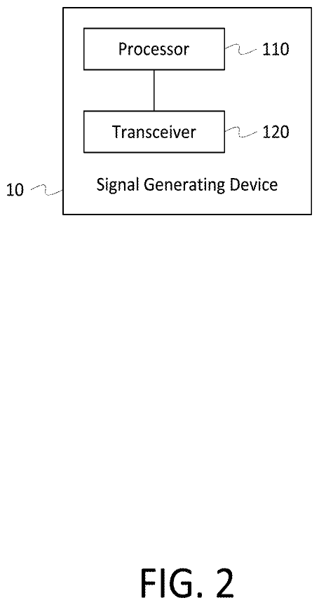

FIG. 2 is a block diagram illustrating the signal generating device 10 according to an example implementation of the present disclosure.

A processor 110 may be also referred to as a controller 110 in the present disclosure. In some implementations, the processor 110 may be, for example, a central processing unit (CPU) or another programmable general-purpose or special-purpose microprocessor, a digital signal processor (DSP), a programmable controller, an application-specific integrated circuit (ASIC), a programmable logic device (PLD), or other similar components or a combination of the components. In addition, the signal generating device 10 may include a memory (not depicted in FIG. 2) storing computer-executable instructions that, when executed by the processor 110, cause the signal generating device 10 to perform various operations described in the present disclosure.

In some implementations, the transceiver 120 may, for example, include a transmitter (e.g., transmitting/transmission circuitry) and a receiver (e.g., receiving/reception circuitry) and may be configured to transmit and/or receive signals (e.g., in a specific frequency band). The transceiver 120 may be controlled by the processor 110 and communicate with the electrode controlling device 20 of the nerve stimulation system 1.

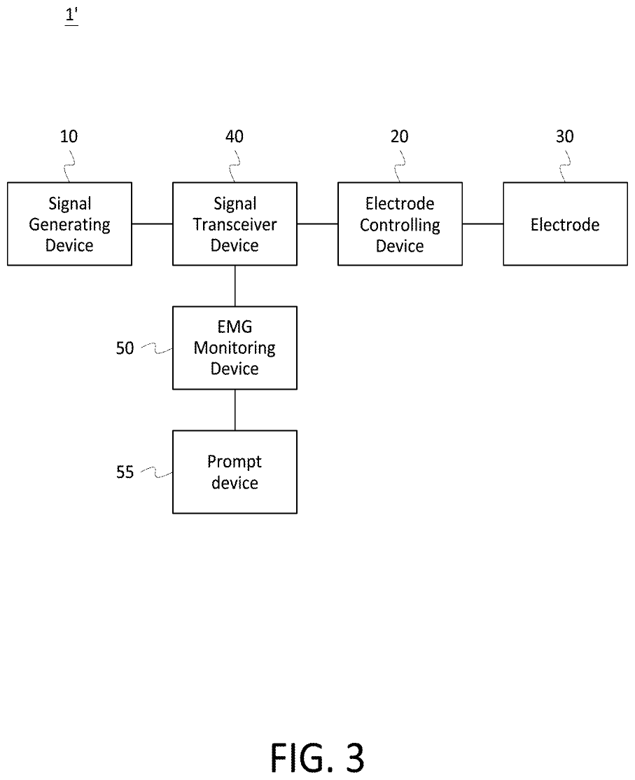

FIG. 3 is a block diagram illustrating a nerve stimulation system according to another example implementation of the present disclosure.

Referring to FIG. 3, a nerve stimulation system 1′ is introduced. In some implementations, the signal generating device 10 may communicate with the electrode controlling device 20 via a signal transceiver device 40. The signal transceiver device 40 may be, for example, configured to relay the nerve stimulation signal from the signal generating device 10 to the electrode controlling device 20.

In some implementations, the signal transceiver device 40 or the signal generating device 10 may further coupled to an electromyography (EMG) monitoring device 50. The EMG monitoring device 50 may be, for example, configured to acquiring an EMG signal from the peripheral nerve, associated with the muscle, that is stimulated by the electrode 30.

In some implementations, the nerve stimulation system 1′ may further includes a prompt device 55 configured to provide a prompt to a user. The prompt device 55 may couple to an EMG monitoring device 50. The prompt device 55 may include, for example, a display, a touch panel, a speaker, an LED, or other similar components or a combination of the components, which is not limited herein.

In some implementations, the signal transceiver device 40 may tune an amplitude of the nerve stimulation signal before relaying it. For example, the signal transceiver device 40 may receive an EMG signal from the EMG monitoring device 50 and tune the amplitude of the nerve stimulation signal according to a voltage of the EMG signal.

In some implementations, the signal generating device 10 may tune an amplitude of the nerve stimulation signal according to feedback from the EMG monitoring device 50. For example, the signal transceiver device 40 may receive an EMG signal from the EMG monitoring device 50 and tune the amplitude of the nerve stimulation signal according to a voltage of the EMG signal.

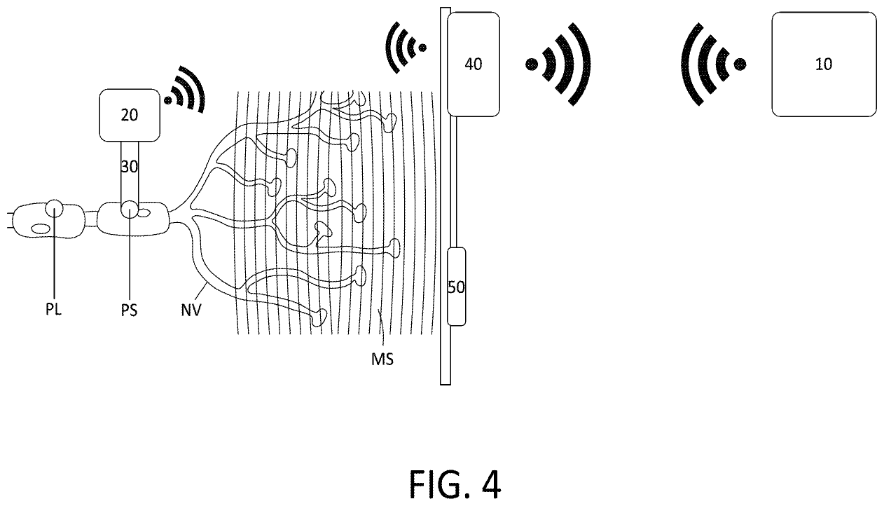

FIG. 4 is a schematic diagram illustrating a nerve stimulation system in operation according to an example implementation of the present disclosure.

Referring to FIG. 4, in some implementations, the electrode controlling device 20 and the electrode 30 may be implanted into a patient and the electrode 30 may aim at a point PS on a peripheral nerve NV, where the point PS is located between a lesion PL of the peripheral nerve NV and a muscle MS associated with the peripheral nerve NV.

In some implementations, a lesion PL of the peripheral nerve NV may cause dysfunction or impairment in the corresponding muscles or sensory areas, resulting in motor or sensory deficits. A lesion PL of the peripheral nerve NV may be caused by acute/chronic nerve injury, such as nerve crush/traction/transection, carpal tunnel syndrome (CTS), muscular dystrophy or neuromuscular disease.

In some implementations, the muscle MS associated with the peripheral nerve NV refers to the muscle MS that is innervated and controlled by peripheral nerves NV. The muscle receives nerve signals and generates corresponding motor responses.

In some implementations, in operation, the signal generating device 10 may generate a nerve stimulation signal and transmit the nerve stimulation signal to the electrode controlling device 20 (e.g., via the signal transceiver device 40). The electrode controlling device 20 may control the electrode 30 to electrically stimulate the peripheral nerve NV at the point PS according to the nerve stimulation signal.

In some implementations, in operation, the EMG monitoring device 50 may acquire an EMG signal associated with the muscle MS and transmit the EMG signal to the signal generating device 10 or the signal transceiver device 40. As such, the signal generating device 10 or the signal transceiver device 40 may tune the amplitude of the nerve stimulation signal transmitted to the electrode controlling device 20 according to a voltage of the EMG signal.

In some implementations, the signal transceiver device 40 or the signal generating device 10 may determine whether a voltage of the EMG signal is within a voltage defined by a maximum threshold (for example, but not limited to, 800 μV) and a minimum threshold (for example, but not limited to, 200 μV).

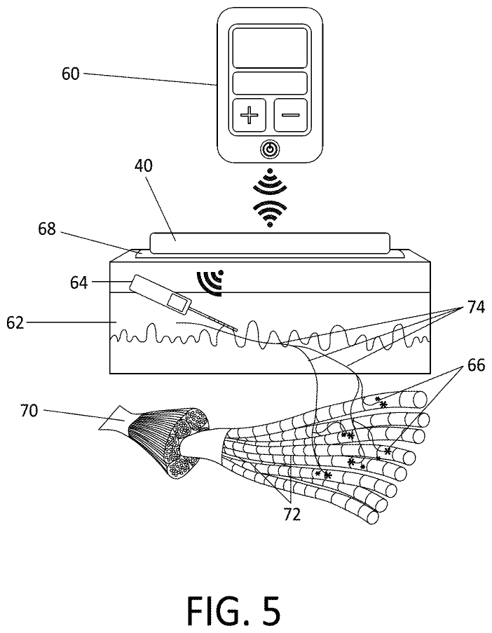

FIG. 5 is a schematic diagram illustrating a nerve stimulation system in operation according to another example implementation of the present disclosure.

Referring to FIG. 5 and FIG. 4, specifically in FIG. 5, muscle 70 is composed of muscle fibers 72, which are the basic structural units of muscle 70. Muscle fibers 72 are connected to motor neurons 74 through neuromuscular junctions 66. The neuromuscular junction 66, which is responsible for transmitting nerve impulses to the muscle 70 and causing muscle contraction and movement, is the point of connection between a motor neuron 74 and a muscle fiber 72. In some implementations, the signal generating device 10 may be a programmer 60 with software, the signal transceiver device 40 may be an external pulse transmitter (EPT), and the electrode controlling device 20 and the electrode 30 may be integrated into an implanted electrode 64. The implanted electrode 64 is implanted to provide electrical stimulation to a target (e.g., a point on the peripheral nerve) distal to an injured point of the peripheral nerve, nearby or close to the nerve innervation so as to target muscle or neuromuscular junction 66. The electrical stimulation may be, for example, treated as an adjunction to other modes of therapy (e.g., medications).

In some implementations, in operation, the signal transceiver device 40 (e.g., the EPT) may be mounted on a skin patch 68 (e.g., disposable hydrogel electrode patch) adhered to a patient's skin 62. The signal generating device 10 may generate a nerve stimulation signal or a waveform of the nerve stimulation signal, and transmit the same to the signal transceiver device 40 (e.g., the EPT). The signal transceiver device 40 (e.g., the EPT) may then transmit the nerve stimulation signal to the electrode controlling device 20. The electrode controlling device 20 may receive the nerve stimulation signal that transmitted percutaneously by the signal transceiver device 40 (e.g., the EPT), then control the electrode 30 to electrically stimulate the target (e.g., the point distal to the injured point of the peripheral nerve).

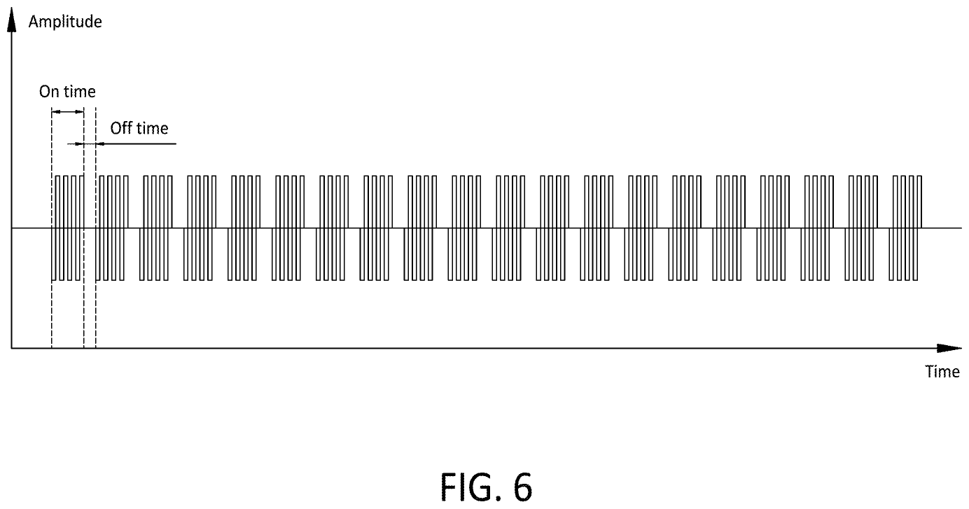

FIG. 6 is a schematic diagram illustrating a waveform of a nerve stimulation signal according to an example implementation of the present disclosure.

Referring to FIG. 6, as mentioned above, the recycle of the Choline and the reproduction of the ACh may take some time. Therefore, an off-time period (e.g., marked as Off time in FIG. 6) is designed in the waveform of the nerve stimulation signal. Several designs of the waveform of the nerve stimulation signal are exemplary described below.

In some implementations, as shown in FIG. 6, the nerve stimulation signal may be a signal with a square envelope, the square envelope periodically includes several on-time periods (e.g., marked as On time in FIG. 6) and off-time periods. The nerve stimulation signal within the on-time period may be an oscillation or repetitive signal with a frequency, e.g., 20 Hz, while the nerve stimulation signal within the off-time period may be, for example, a zero signal. That is, the nerve stimulation signal may be a signal with a pulse amplitude within the on-time period and may be a signal without the pulse amplitude within the off-time period. A ratio of an on-time period and an off-time period is not less than 1. More specifically, an off-time period may not be longer than an on-time period. It is noted that the frequency of the oscillation signal may be also referred to as stimulation frequency in the present disclosure.

In some implementations, a ratio of the on-time period and the off-period may be designed as 4. For example, a length of the on-time period may be 8 seconds, and a length of the off-time period may be 2 seconds, but which is not limited herein.

In some implementations, a length of the off-time period may be not longer than 5 seconds.

In some implementations, a length of the off-time period may be limited within a range of 1 second to 5 seconds. In a case that the ratio of the on-time period and the off-time period is designed as 4, the on-time period should be within a range of 4 seconds to 20 seconds. In some implementations, a length of the off-time period may be limited within a range of 0.5 second to 5 seconds. In a case that the ratio of the on-time period and the off-time period is designed as 4, the on-time period should be within a range of 2 seconds to 20 seconds.

In some implementations, a length of the off-time period may be 2 seconds. In a case that the ratio of the on-time period and the off-time period is designed as 4, the on-time period should be 8 seconds.

In some implementations, a length of the on-time period may be limited within a range of 5 seconds to 9 seconds. In a case that the ratio of the on-time period and the off-time period is designed as 4, the off-period should be within a range of 1.25 seconds to 2.25 seconds.

In some implementations, the duty cycle of the on-time period and the off-time period is within a range of 60% to 80%, but which is not limited herein. In addition, said an 80% duty cycle means the signal is on 80% of the time but off 20% of the time.

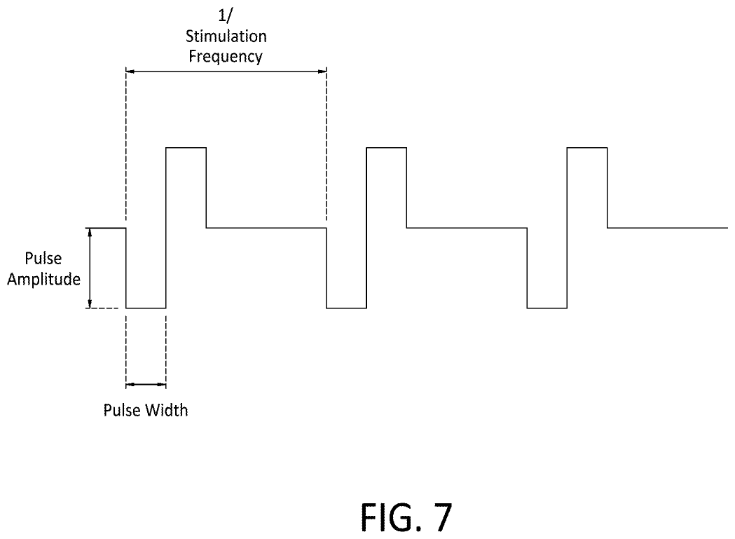

FIG. 7 is a schematic diagram illustrating a waveform of a nerve stimulation signal in the on-time period according to an example implementation of the present disclosure.

Referring to FIG. 7, within the on-time period, the waveform of the oscillation or repetitive signal in one period may, for example, sequentially include a negative pulse with a pulse width and a positive pulse with the same pulse width followed by a zero signal. In addition, said one period may be (1/stimulation frequency) seconds. However, details of the waveform are not limited in the present disclosure.

In some implementations, the stimulation frequency of the nerve stimulation signal within the on-time period may be within a range of 5 Hz to 500 Hz. For example, the stimulation frequency may be 20 Hz. More specifically, within an on-time period, a negative/positive pulse may arrive for each 0.05 seconds in a case that the stimulation frequency is 20 Hz.

In some implementations, the stimulation frequency of the nerve stimulation signal within the on-time period may be within a range of 5 Hz-10 Hz, 10 Hz-Hz, 15 Hz-20 Hz, 20 Hz-30 Hz, 30 Hz-40 Hz, 40 Hz-50 Hz, 50 Hz-60 Hz, 60 Hz-70 Hz, Hz-80 Hz, 80 Hz-90 Hz, 90 Hz-100 Hz, 100 Hz-110 Hz, 110 Hz-120 Hz,120 Hz-130 Hz, 130 Hz-140 Hz, 140 Hz-150 Hz, 150 Hz-160 Hz, 160 Hz-170 Hz, 170 Hz-180 Hz, 180 Hz-190 Hz, 190 Hz-200 Hz, 200 Hz-250 Hz, 250 Hz-300 Hz, 300 Hz-350 Hz, 350 Hz-400 Hz, 400 Hz-450 Hz, or 450 Hz-500 Hz.

In some implementations, the positive pulse amplitude is within a range of mA to 0.5 mA, and the negative pulse amplitude is within a range of −0.1 mA to −0.5 mA.

In some implementations, the positive pulse amplitude may be, for example but not limited to, 1 mA, and the negative pulse amplitude may be, for example but not limited to, −1 mA.

In some implementations, the positive pulse amplitude may be, for example but not limited to, 0.4 mA, and the negative pulse amplitude may be, for example but not limited to, −0.4 mA.

In some implementations, the pulse width is within a range of 100 μs to 500 μs.

In some implementations, the pulse width is within a range of 100 μs to 200 μs.

In some implementations, the pulse width may be, for example but not limited to, 200 μs.

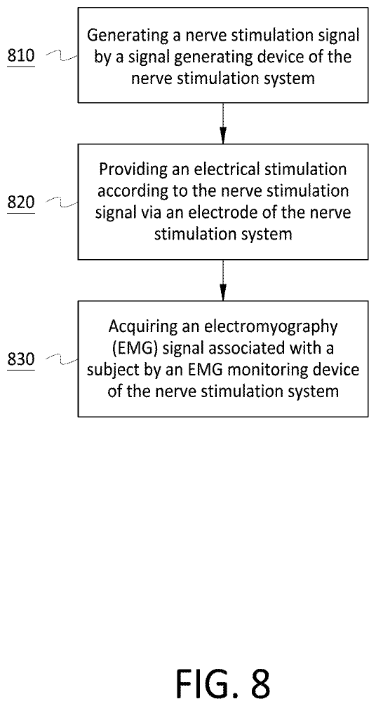

FIG. 8 is a flowchart illustrating a method for operation of a nerve stimulation according to an example implementation of the present disclosure.

Referring to FIG. 8, in action 810, a nerve stimulation signal may be generated. The signal generating device 10 may communicate with the electrode controlling device 20 via a signal transceiver device 40. The signal transceiver device 40 may be, for example, configured to relay the nerve stimulation signal from the signal generating device 10 to the electrode controlling device 20.

In some implementations, the signal generating device 10 may generate a nerve stimulation signal and transmit the nerve stimulation signal to the electrode controlling device 20 (e.g., via the signal transceiver device 40).

Returning to FIG. 8, in action 820, an electrode 30 may be provided and the electrode 30 may aim at a peripheral nerve. The generated nerve stimulation signal may be transmitted to the electrode controlling device 20 such that the electrode controlling device 20 may control the electrode 30 according to the received nerve stimulation signal.

In some implementations, the peripheral nerve (e.g., NV as depicted in FIG. 4) may be injured at a point (e.g., PL as depicted in FIG. 4). The electrode 30 may aim at a point (e.g., PS as depicted in FIG. 4) located on the injured peripheral nerve and between the lesion (e.g., PL as depicted in FIG. 4) of the peripheral nerve and a muscle (e.g., MS as depicted in FIG. 4) associated with the injured peripheral nerve.

In some implementations, the provided electrode 30 may be, for example, implanted into a subject (e.g., patient) for the treatment.

Returning to FIG. 8, in action 830, acquiring an electromyography (EMG) signal associated with a subject by an EMG monitoring device 50 of the nerve stimulation system.

In some implementations, the electrode controlling device 20 may control the electrode 30 to electrically stimulate the peripheral nerve according to the nerve stimulation signal.

In some implementations, after the electrode 30 starts to stimulate the peripheral nerve, the signal transceiver device 40 or the signal generating device 10 may acquire, from the EMG monitoring device 50, an EMG signal associated with a subject (e.g., the muscle associated with the stimulated nerve). The signal transceiver device 40 or the signal generating device 10 may determine whether a voltage of the EMG signal is within a voltage defined by a maximum threshold (for example, but not limited to, 800 μV) and a minimum threshold (for example, but not limited to, 200 μV).

In a case of determining that the voltage of the EMG signal is greater than the maximum threshold, which means that an excessive current may occur when stimulating the nerve, the signal transceiver device 40 or the signal generating device 10 may decrease an amplitude of the nerve stimulation signal for electrically stimulating the peripheral nerve. In some implementations, a prompt device of the nerve stimulation system 1 or 1′ may provide a prompt indicating an excessive current. In a case of determining that the voltage of the EMG signal is within the voltage range, a prompt device of the nerve stimulation system 1′ may provide a prompt indicating an efficient stimulation is undergoing.

In a case of determining that the voltage of the EMG signal is less than the minimum threshold, which means that an insufficient current may occur when stimulating the nerve, the signal transceiver device 40 or the signal generating device 10 may increase an amplitude of the nerve stimulation signal for electrically stimulating the peripheral nerve. In some implementations, a prompt device of the nerve stimulation system 1′ may provide a prompt indicating an insufficient current.

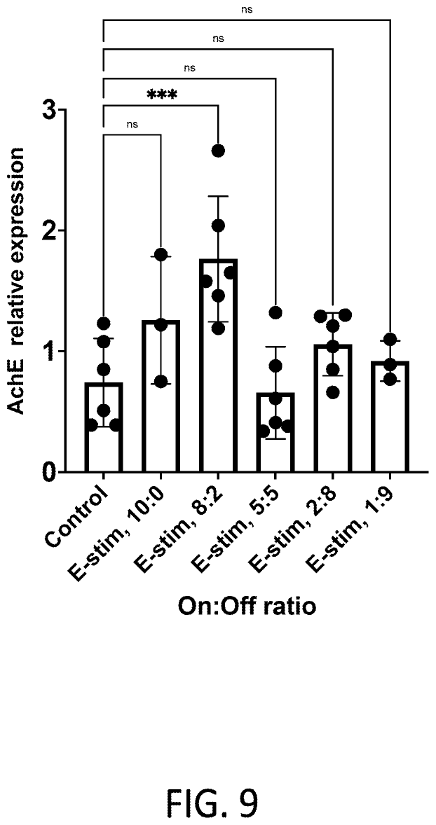

FIG. 9 illustrates the AchE relative expressions with different on:off ratios according to an example implementation of the present disclosure.

The on:off ratio may be the ratio of the on-time period and the off-time period. In a case of an on:off ratio is designed as 8:2, the on-time period may be 2 seconds and the off-period may be 0.5 seconds.

In order to determine the AchE relative expressions with different on:off ratios, an enzyme-linked immunosorbent assay (ELISA) is performed. First, the sciatic nerve of each rat was injured. Then, different on:off ratios of the electrical nerve stimulation is applied. After that, denervated gastrocnemius was harvested after euthanasia of rats. For each rat, muscle specimen was harvested at the insertion of sciatic nerve to muscle, followed by tissue homogenization according to the instructions. Briefly, 50 mg tissues were homogenized in 0.5 mL of 1X PBS, pH 7.4 and stored overnight at −20° C., following two freeze-thaw cycles to break the cell membranes. Then the homogenate was centrifuged for 5 minutes at 5,000×g, 4° C., and the total protein concentration of all samples was performed by using the Pierce BCA protein assay kit (Thermo Scientific, Rockford, Ill., USA). Finally, quantitative measurement of the concentration of AchE was conducted using an ELISA assay kit (OKEH03313, San Diego, Calif., USA) and the absorbance was then measured at 450 nm by using a microplate reader. Moreover, in FIG. 9, the control means a rat with the injured sciatic nerve without applying any electrical nerve stimulation after the nerve damage.

Acetylcholine (Ach) will be released from the axon terminal and accepted by the muscle MS associated with the peripheral nerve NV when the electrode 30 electrically stimulates the peripheral nerve NV at the point PS according to the nerve stimulation signal. After electrically stimulated with different on:off ratios, the muscle MS is then extracted and the expression of AchE is further examined by the aforementioned method. The more expression of AchE is indicative of a more effective ACh synthesis pathway. Which means the recycle of the Choline and the reproduction of the Ach is more effective. As shown in FIG. 9, the expression of AchR is significantly higher when the electrode 30 electrically stimulates the peripheral nerve NV at the point PS is stimulated with an on/off ratio of 8:2 compared to other on/off ratios including 10:0, 5:5, 2:8, 1:9, and the control. Therefore, the results show that applying the electrical nerve stimulation with on/off ratio of 8:2 is a more effective way for treating nerve injury than the other on/off ratios including 10:0, 5:5, 2:8, 1:9 for treating nerve injury.

In order to determine the aAChR and myo D mRNA relative expression with different on:off ratios, a quantitative PCR is performed. First, total RNA was extracted from denervated gastrocnemius tissues of rat with TRIzol, according to the instructions. Total RNA was precipitated with 2-propanol, washed twice with 75% ethanol, and resuspended in a suitable volume of DEPC-treated water. The total RNA concentration was determined by measuring the OD values of the samples at 260 nm. To prepare first strand cDNA, mRNA was reserve transcribed with reverse transcriptase enzyme (ImProm-II™ Reverse Transcriptase, Madison, Wis., USA) in a total 20 ml reaction mixture.

Primers were designed for aAchR, myoD and glyceraldehyde 3-phosphate dehydrogenase (GAPDH). GAPDH was used as a reference gene. Primer sequences of aAchR, myoD and GAPDH are shown in Table 1. To analyze mRNA level, quantitative PCR was performed with SYBR PCR Master Mix (GoTaq® Green Master Mix, Madison, Wis., USA) and StepOnePlus™ System (Applied Biosystems, Calif.). The quantitative PCR conditions were as follows: 10 min at 95° C., followed by 40 cycles of 15 sec at 95° C. and 30 sec at 60° C. The 2^{-ΔΔCT} method was used to analyze quantitative PCR data.

TABLE. 1
Primer sequences of αAchR, myoD and GAPDH.

| Name | Forward primer/reverse primer |
|---|---|
| αAChR | 5′-TCCCTTCGATGAGCAGAACT-3′ |
| | 5′-ATCACCCACTCTCCGCTCT-3′ |
| myoD | 5-GGAGACATCCTCAAGCGATGC-3′ |
| | 5-AGCACCTGGTAAATCGGATTG-3′ |
| GAPDH | 5′-TGGCCTCCAAGGAGTAAGAA-3′ |
| | 5′-TGTGAGGGAGATGCTCAGTG-3′ |

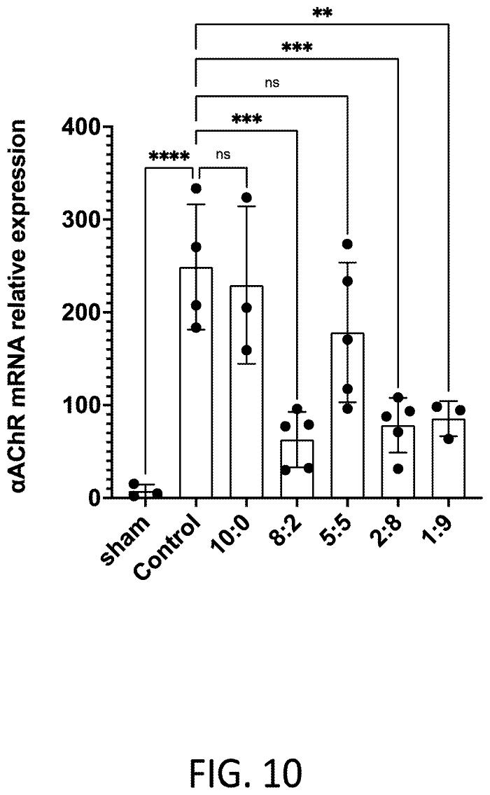

FIG. 10 illustrates the aAChR mRNA relative expressions with different on:off ratio ratios according to an example implementation of the present disclosure.

The electrode 30 electrically stimulates the peripheral nerve NV at the point PS according to the nerve stimulation signal. After electrically stimulated with different on:off ratios, the muscle MS is then extracted and the expression of aAChR mRNA is further examined by the aforementioned method. When the degree of denervated muscle injury increases, the relative expression level of aAChR mRNA increases. As shown in

FIG. 10, the lower expression of aAChR mRNA is indicative of a less degree of the nerve/muscle injury. Moreover, in FIG. 10, the control indicates a rat with the injured sciatic nerve without applying any electrical nerve stimulation after the nerve damage, while sham indicates a sham-operated rat, and the sciatic nerves of the rat are not injured. The expression of aAChR mRNA is significantly lower when the electrode 30 electrically stimulates the peripheral nerve NV at the point PS is stimulated with an on/off ratio of 8:2 compared to other on/off ratios including 10:0, 5:5, 2:8, 1:9, and the control. Therefore, the results show that applying the electrical nerve stimulation with on/off ratio of 8:2 is a more effective way for treating nerve/muscle injury than the other on/off ratios including 10:0, 5:5, 2:8, 1:9 for treating nerve/muscle injury.

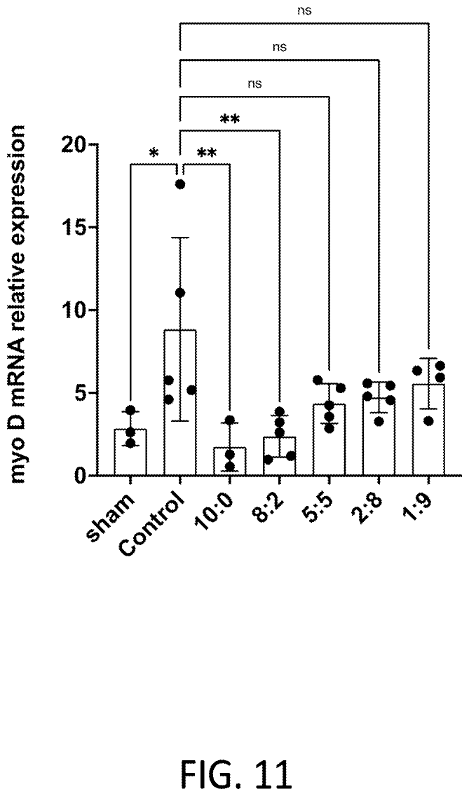

FIG. 11 illustrates the myo D mRNA relative expressions with different on:off ratios according to an example implementation of the present disclosure.

The electrode 30 electrically stimulates the peripheral nerve NV at the point PS according to the nerve stimulation signal. After electrically stimulated with different on:off ratios, the muscle MS is then extracted and the expression of myo D mRNA is further examined by the aforementioned method. When the degree of denervated muscle injury increases, the relative expression level of myo D mRNA increases. As shown in FIG. 10, the lower expression of myo D mRNA is indicative of a less degree of the nerve/muscle injury. Moreover, in FIG. 10, the control indicates a rat with the injured tibial nerve without applying any electrical nerve stimulation after the nerve damage, while sham indicates a sham-operated rat, and the nerves of the rat are not injured. In FIG. 10, the expression of myo D mRNA is significantly lower when the electrode 30 electrically stimulates the peripheral nerve NV at the point PS is stimulated with an on/off ratios of 8:2 or 10:0 compared to other on/off ratios including 5:5, 2:8, 1:9, and the control. Therefore, the results show that applying the electrical nerve stimulation with on/off ratio of 8:2 is a more effective way for treating nerve/muscle injury than the other on/off ratios including 5:5, 2:8, 1:9 for treating nerve/muscle injury.

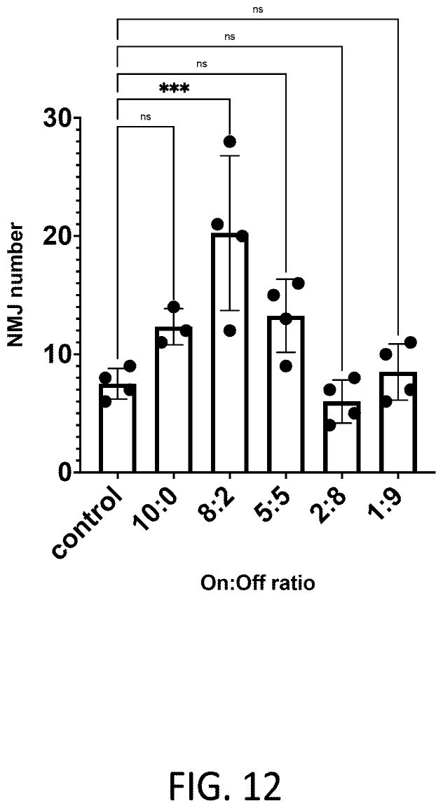

FIG. 12 illustrates the number of neuromuscular junctions (NMJ) with different on:off ratios according to an example implementation of the present disclosure.

In order to determine the number of neuromuscular junctions (NMJ) with different on:off ratios, an immunofluorescent neuromuscular junction staining is performed. After euthanasia, denervated gastrocnemius muscles were harvested and fixed in Optical Coherence Tomography (OCT) for cryosection. The tissue sections of each were 40 μm. For immunofluorescent staining, the tissue sections were labeled using a rabbit anti-NF200 antibody (1:200; Abcam) overnight at 4° C. After washing three times with phosphate buffered saline (PBS), sections were incubated with Alpha-Bungarotoxin Conjugates conjugated Alexa Fluor™ 594 (1:200; Thermo Fisher Scientific) and for 1 hour at room temperature. Image stacks were taken by the FLUOVIEW FV3000 Laser Scanning Microscope.

The electrode 30 electrically stimulates the peripheral nerve NV at the point PS according to the nerve stimulation signal. After electrically stimulated with different on:off ratios, the muscle MS is then prepared for cryosection and immunofluorescent neuromuscular junction staining is further examined by the aforementioned method. When the degree of denervated muscle injury increases, the number of neuromuscular junction decreases. As shown in FIG. 12, the lower number of NMJ is indicative of a less degree of the nerve/muscle injury. Moreover, in FIG. 12, the control indicates a rat with the injured tibial nerve without applying any electrical nerve stimulation after the nerve damage. The number of neuromuscular junction is significantly higher when the electrode 30 electrically stimulates the peripheral nerve NV at the point PS is stimulated with an on/off ratio of 8:2 compared to other on/off ratios including 10:0, 5:5, 2:8, 1:9, and the control. Therefore, the results show that applying the electrical nerve stimulation with on/off ratio of 8:2 is a more effective way for treating nerve/muscle injury than the other on/off ratios including 10:0, 5:5, 2:8, 1:9 for treating nerve/muscle injury.

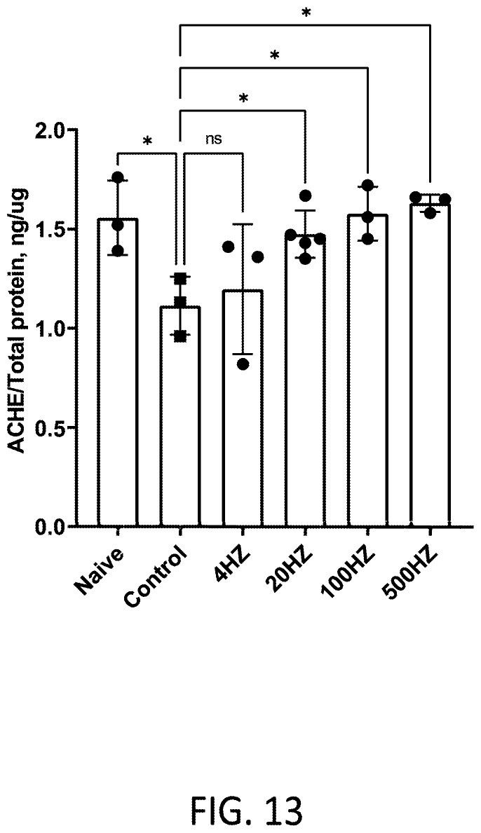

FIG. 13 illustrates the AchE/Total proteins with different stimulation frequencies according to an example implementation of the present disclosure.

In order to determine the AchE/Total proteins with different stimulation frequencies, an ELISA is performed. First, the sciatic nerve of each rat was injured. Then, different stimulation frequencies of the electrical nerve stimulation are applied. After that, denervated gastrocnemius was harvested after euthanasia of rats. For each rat, muscle specimen was harvested at the insertion of sciatic nerve to muscle, followed by tissue homogenization according to the instructions. Briefly, 50 mg tissues were homogenized in 0.5 mL of 1X PBS, pH 7.4 and stored overnight at −20° C., following two freeze-thaw cycles to break the cell membranes. Then the homogenate was centrifuged for 5 minutes at 5,000×g, 4° C., and the total protein concentration of all samples was performed by using the Pierce BCA protein assay kit (Thermo Scientific, Rockford, Ill., USA). Finally, quantitative measurement of the concentration of AchE was conducted using an ELISA assay kit (OKEH03313, San Diego, Calif., USA) and the absorbance was then measured at 450 nm using a microplate reader. Moreover, in FIG. 13, the control indicates a rat with the injured tibial nerve without applying any electrical nerve stimulation after the nerve damage; naive means it is a non-operated rat.

Acetylcholine (Ach) will release from the axon terminal and accepted by the muscle MS associated with the peripheral nerve NV when the electrode 30 electrically stimulates the peripheral nerve NV at the point PS according to the nerve stimulation signal. After electrically stimulated with different stimulation frequencies, the muscle MS is then extracted, and the AchE/total protein is further examined by the aforementioned method. The more of AchE/total protein is indicative of a more effective ACh synthesis pathway. Which means the recycle of the Choline and the reproduction of the Ach is more effective. As shown in FIG. 13, the AchE/total protein is significantly higher when the electrode 30 electrically stimulates the peripheral nerve NV at the point PS is stimulated with stimulation frequencies of 20 Hz, 100 Hz and 500 Hz compared to other different stimulation frequency including 4 Hz, and the control. Therefore, the results show that applying the electrical nerve stimulation with stimulation frequencies of 20 Hz, 100 Hz and 500 Hz is a more effective way for treating nerve injury than the 4 Hz stimulation frequency for treating nerve injury.

In some implementations, the nerve stimulation system/device shows the effective way for treating nerve injury when the frequency is less than or equal to 500 Hz and more than 4 Hz. Thus, a stimulation frequency may be applied within a range of 5 Hz to 500 Hz.

As aforementioned, the nerve stimulation system/device shows the effective way for treating nerve injury when stimulated with an on/off ratio of 8:2 and a stimulation frequency within a range of 5 Hz to 500 Hz.

For the application of the nerve stimulation system/device, the nerve stimulation system/device may be used to treat acute/chronic nerve damage (e.g., carpal tunnel syndrome) or muscle degenerative diseases (e.g., congenital muscular dystrophy).

The embodiments shown and described above are only examples. Many details are often found in the art. Therefore, many such details are neither shown nor described. Even though numerous characteristics and advantages of the present disclosure have been set forth in the foregoing description, together with details of the structure and function of the present disclosure, the present disclosure is illustrative only, and changes may be made in the details. It will therefore be appreciated that the embodiment described above may be modified within the scope of the claims.

What is claimed is:

1. A nerve stimulation system, comprising:
an electrode;
an electrode controlling device coupled to the electrode and configured to control the electrode to electrically stimulate a peripheral nerve according to a nerve stimulation signal;
and a signal generating device coupled to the electrode controlling device and configured to generate the nerve stimulation signal, wherein the nerve stimulation signal is a signal with a square envelope, the square envelope periodically comprises an on-time period with a pulse amplitude and an off-time period without the pulse amplitude, a ratio of the on-time period and the off-time period is not less than 1, and a length of the off-time period is not longer than 5 seconds.

2. The nerve stimulation system according to claim 1, wherein the electrode is to be implanted and aimed at a point between a lesion of the peripheral nerve and a muscle associated with the peripheral nerve.

3. The nerve stimulation system according to claim 1, further comprising:
an electromyography (EMG) monitoring device coupled to the signal generating device and configured to acquire an EMG signal associated with a muscle, wherein:
in a case that a voltage of the EMG signal is greater than a maximum threshold, the signal generating device is further configured to decrease an amplitude of the nerve stimulation signal for electrically stimulating the peripheral nerve, and in a case that the voltage of the EMG signal is less than a minimum threshold, the signal generating device is further configured to increase the amplitude of the nerve stimulation signal for electrically stimulating the peripheral nerve.

4. The nerve stimulation system according to claim 3, wherein the maximum threshold is 800 μV.

5. The nerve stimulation system according to claim 3, wherein the minimum threshold is 200 μV.

6. The nerve stimulation system according to claim 1, further comprising:
a prompt device coupled to an EMG monitoring device, wherein:
in a case that a voltage of an EMG signal is greater than a maximum threshold, the prompt device is configured to provide a prompt indicating an excessive current;
in a case that the voltage falls within a voltage range defined by the maximum threshold and a minimum threshold, the prompt device is configured to provide the prompt indicating an efficient stimulation; and
in a case that the voltage of the EMG signal is less than the minimum threshold, the prompt device is configured to provide the prompt indicating an insufficient current.

7. The nerve stimulation system according to claim 1, wherein the length of the off-time period is within a range from 0.5 second to 5 seconds.

8. The nerve stimulation system according to claim 1, wherein the ratio of the on-time period to the off-time period is 4.

9. The nerve stimulation system according to claim 1, wherein a length of the on-time period is with a range from 3 seconds to 9 seconds.

10. The nerve stimulation system according to claim 1, wherein a frequency of the nerve stimulation signal in the on-time period is within a range of 5 Hz to 500 Hz.

11. The nerve stimulation system according to claim 1, wherein a duty cycle of the on-time period and the off-time period is within a range of 60% to 80%.

12. A signal generating device, comprising:
a transceiver coupled to an electrode controlling device; and
a processor coupled to a transceiver and configured to:
generate a nerve stimulation signal; and
transmit, using the transceiver, the nerve stimulation signal to the electrode controlling device such that the electrode controlling device controls an electrode to electrically stimulate a peripheral nerve according to the nerve stimulation signal,
wherein the nerve stimulation signal is a signal with a square envelope, the square envelope periodically comprises an on-time period with a pulse amplitude and an off-time period without the pulse amplitude, a ratio of the on-time period and the off-time period is not less than 1, and a length of the off-time period is not longer than 5 seconds.

13. The signal generating device according to claim 12, wherein the electrode is to be implanted and aiming at a point between a lesion of the peripheral nerve and a muscle associated with the peripheral nerve.

14. The signal generating device according to claim 12, wherein the processor is further configured to:
receive an electromyography (EMG) signal associated with a muscle from an EMG monitoring device by using the transceiver;
determine whether a voltage of the EMG signal falls within a voltage range defined by a maximum threshold and a minimum threshold;
in a case of determining that the voltage of the EMG signal is greater than the maximum threshold, decrease an amplitude of the nerve stimulation signal; and
in a case of determining that the voltage of the EMG signal is less than the minimum threshold, increase the amplitude of the nerve stimulation signal.

15. The signal generating device according to claim 12, wherein a frequency of the nerve stimulation signal in the on-time period is within a range of 5 Hz to 500 Hz.

16. The signal generating device according to claim 12, wherein the ratio of the on-time period to the off-time period is 4.

17. An operation method of a nerve stimulation system comprising:
generating a nerve stimulation signal by a signal generating device of the nerve stimulation system;
providing an electrical stimulation according to the nerve stimulation signal via an electrode of the nerve stimulation system;
acquiring an electromyography (EMG) signal associated with a subject by an EMG monitoring device of the nerve stimulation system; and
determining whether a voltage of the EMG signal falls within a voltage range defined by a maximum threshold and a minimum threshold,
wherein the nerve stimulation signal is a signal with a square envelope, the square envelope periodically comprises an on-time period with a pulse amplitude and an off-time period without the pulse amplitude, a ratio of the on-time period and the off-time period is not less than 1, and a length of the off-time period is not longer than 5 seconds.

18. The operation method according to claim 17, wherein the length of the off-time period is within a range from 0.5 second to 5 seconds.

19. The operation method according to claim 17, wherein the length of the off-time period is 2 seconds.

20. The operation method according to claim 17, wherein the ratio of the on-time period to the off-time period is 4.
